(19) United States
(12) Patent Application Publication
(10) Pub. No.: US 2021/0008215 A1
(43) Pub. Date: Jan. 14, 2021

(54) OPTIMIZING DRUG DELIVERY

(71) Applicant: Henry J. Smith, Temecula, CA (US)

(72) Inventor: Henry J. Smith, Temecula, CA (US)

(21) Appl. No.: 16/505,474

(22) Filed: Jul. 8, 2019

(51) Int. Cl. A61K 47/24; A61K 9/107

(52) U.S. Cl. CPC A61K 47/24; B82Y 5/00; A61K 9/1075

(57) ABSTRACT

This invention teaches a method of increasing the bioavailability, safety and efficacy of a cancer drug incorporated in a nanocarrier such as liposomes, micelles, dendrimeres, nanoemulsion, nanoparticles and antibody drug conjugates. It does so by administering pre-blocking blank liposomes to the patient several hours before the drug incorporated nanocarrier is administered. Blocking the reticuloendothelial system (RES) will prevent it from taking up the drug incorporated nanocarrier and hence improve the safety and efficacy of the drug.

### CROSS-REFERENCE TO RELATED APPLICATIONS

Not Applicable.

### STATEMENT REGARDING FEDERALLY SPONSORED RESEARCH OR DEVELOPMENT

Not Applicable

### BACKGROUND INFORMATION

There are a large number of small molecule cancer drugs. They are effective in inhibiting tumor growth but almost all are known to have serious side-effects. Many studies have shown that incorporating these drugs into a variety of nanosized drug delivery vehicles (“nanocarriers”) such as liposomes, micelles, dendrimers, nanoparticles, lipid nanospheres, nanocapsules, nanoemulsions, and antibody drug conjugates could improve the safety and efficacy of the drug. For example, incorporating the small molecule drug into liposomes or other nanocarriers significantly reduced the adverse side-effects typically associated with administering the free drug. By Incorporating the cancer drug into a carrier this prevented it from exiting the blood stream and causing harm to normal tissue cells. It also shielded the drug from being detoxified by the liver or rapidly excreted out by the kidneys thus making more drug bioavailable to treat the tumor.

Despite these significant improvements it was noted that when the drug nanocarrier was administered intravenously into a host animal a major percentage of the drug nanocarrier was actually being taken up by the reticuloendothelial system (RES) in the liver and spleen, and therefore only a small fraction of the injected dose was available to treat the tumor. There was therefore intensive research focusing on ways of improving the pharmokinetics of the drug nanocarrier so that there would be less uptake by the RES and more available to treat the tumor.

It was discovered that reducing the size of the nanocarrier (e.g. 100 nm or less) and changing its chemical composition by using natural substances (e.g. lecithin) would reduce its uptake by the RES. Also incorporating long chain polymers (e.g. polyethylene glycol) into its surface structure would also shield it from uptake by the RES. This has resulted in a significant improvement in the bioavailability of the drug to treat the tumor.

It is important to note however, that despite these major improvements it appears that a major portion of the injected drug nanocarrier is still being sequestered in the liver and spleen. There is obviously still room for improvement. It would be desirable if there was a way to further improve the safety and bioavailability of the therapeutic drug.

This invention teaches a means of improving the bioavailability, safety and efficacy of a drug. It does so not by changing the nature of the drug and/or the nanocarrier, but instead by blocking the RES so that it is unable to recognize and respond to a subsequent exposure to the therapeutic drug nanocarrier. This invention discloses a liposomal formulation that is expressly designed to pre-block the RES from taking up a variety of therapeutic drug nanocarriers and thus improve their safety and efficacy.

The idea of using liposomes to block the RES is not new. What is novel about this invention however, is that it discloses a liposomal formulation that right from the start is not designed to be a drug delivery system that protects a small molecule cancer drug from being detoxified by the liver or removed by the RES. Instead it teaches a liposomal formulation that ignores these conventional attributes and whose sole function is to block the RES from responding to the therapeutic drug nanocarrier.

### SUMMARY

This invention teaches a method of increasing the bioavailability, safety and efficacy of a cancer drug incorporated in a nanocarrier such as liposomes, micelles, dendrimeres, nanoemulsion, nanoparticles and antibody drug conjugates. It does so by administering pre-blocking blank liposomes to the patient several hours before the drug incorporated nanocarrier is administered. Blocking the reticuloendothelial system (RES) will prevent it from taking up the drug incorporated nanocarrier and hence improve the safety and efficacy of the drug.

### DETAILED DESCRIPTION OF THE INVENTION

Ever since the discovery of lipid vesicles by Bangham et al. (1965) there has been great interest about the possibility of using liposomes as a delivery system for small molecule cancer drugs (Gregoriadis G., Ryman B. E. 1971; Gregoriadis G. 1976). The advantages of a liposomal drug are obvious. The ability to protect the drug from being filtered out by the kidneys or detoxified by the liver will increase in bioavailability. The ability to prevent the drug from extravasating into normal tissues and causing harm would be of great benefit to patients experiencing the very serious side-effects of cancer chemotherapy. Yet almost 50 years after liposomes were first proposed as promising drug delivery vehicles there are only a handful of liposomal drugs that have been developed and commercialized.

The reason is that there are in fact many problems associated with developing a liposomal drug. The major problems are their propensity to leak during storage, and when they are administered into the blood stream a major fraction of the liposomal drug was sequestered in the liver and spleen leaving only a minor fraction available to reach the tumor. It was soon realized that when liposomes were administered into the blood they were being quickly recognized and removed by the macrophages and mononuclear phagocytic cells of the reticuloendothelial system (RES) present in the liver and spleen.

In order to mitigate this effect there is intensive ongoing research into developing new liposomal formulations that would prevent their recognition and removal by the RES. Briefly, there several ways this has been done. One way is to reduce the size of the liposomes. Early research demonstrated that small liposomes persisted longer in the blood circulation than larger liposomes. Therefore most formulations of liposomal drugs use liposomes that are standardized to have a uniform diameter that is under 200 nm and often they are sized to be 100 nm or less. Another way to protect the liposomes from being recognized by the RES is to coat them with long chain polymer molecules that would provide steric hindrance from being coated with opsonins and/or contact with the surface of the phagocytic cell. Typically the liposome is coated with long chain polymers of polyethylene glycol having a MW of 2,000 or more. These modifications have resulted in a significant reduction in their uptake by the RES and a corresponding increase of the liposomal drug in the blood. It should be noted however that despite these improvements a major fraction of the liposomal drug is still being recognized by the RES and sequestered in the liver and spleen.

An alternative method of preventing the liposomal drug from being recognized and bound out by the RES is to pre-block the RES with blank (i.e. no drug) liposomes prior to administering the therapeutic liposomal drug. The pre-blocking liposomes will be taken up by macrophages and mononuclear phagocytic cells of the RES which are therefore unable to respond to the later injection of the therapeutic drug liposomes.

The idea of pre-blocking the RES using blank liposomes is not new. Early studies showed that when liposomes were administered intravenously into animals almost all the liposomes quickly disappeared form the blood circulation and became localized in the liver and spleen because of their uptake by the RES (Juliano, R. L. Stamp, D. 1975). One obvious approach to mitigate this problem would be see if pre-blocking the RES with non-drug incorporated liposomes would prevent the RES from responding to a subsequent injection of liposomes. Kao, Y. et al. (1981) reported the interaction of liposomes with the reticuloendothelial system. Abra et al (1981, 1982) described administering different sizes of pre-blocking blank liposomes and noted that pre-blocking reduced the uptake of a subsequent injection of liposomes. Ellens, H., et al. (1982) described the reversible depression of the reticuloendothelial system by liposomes. It is therefore surprising to find that almost 40 years later how little progress has been made to follow up on this promising approach. Liu et al. (2015) reported that commercial liposomes could be used to pre-block the RES and increase the bioavailability of paclitaxel nanoparticles to treat cancer; but to date there are no commercially available pre-blocking liposomes available for use.

This invention teaches a novel stabilized liposomal formulation that is expressly designed to comprehensively pre-block the RES from responding to a variety of different liposomal drugs. Further, that it is also capable of pre-blocking the RES from responding to a variety of other different drug nanocarriers including micelles, dendrimers, nanoemulsions, nanoparticles, and antibody drug conjugates. It teaches the principles underlying this invention and their application; and the reasons why the pre-blocking liposomes of this invention are fundamentally different from conventional liposomes.

In contrast to conventional liposomes that are designed to avoid recognition and removal by the RES the pre-blocking liposomes of this invention are expressly designed to be recognized and taken up by the RES. And while conventional liposomes are designed to encapsulate and retain water-soluble drugs within their aqueous interior the pre-blocking liposomes of this invention are obviously not subject to this restriction.

There are however, certain features to consider in developing an effective pre-blocking liposome formulation. First, the pre-blocking liposomes must be safe to use even when used at high dosages. Second, it must have an efficient blocking capacity so that the effective dosage is kept as low as possible. Third, it should be biodegradable with no residual harmful effects. Fourth, it should be non-immunogenic. Fifth, on a practical basis it should be capable of blocking a variety of drug incorporated nanocarriers including liposomes, micelles, nanoemulsions, dendrimers, lipid nanospheres, nanocapsules and other types of drug incorporated nanoparticles such as antibody drug conjugates (ADC). Finally it should be stable when stored for a prolonged period of time.

The following example is provided to illustrate the basic procedure used to prepare the pre-blocking liposomes. It will be obvious to those of skill in the art that there are many different methods of preparing liposomes. Those that result in a final pre-blocking liposomal product that resembles that disclosed in this invention are considered to lie within the scope and spirit of this invention.

### Example 1

The pre-blocking liposomes are prepared using one or more phospholipids selected from the following list: dioleoyl phosphatidylcholine (DOPC), egg phosphatidylcholine (EPC), hydrogenated egg phosphatidylcholine (HEPC), soy phosphatidylcholine (SPC), hydrogenated soy phosphatidylcholine (HSPC), dimyristoylphosphatidylcholine (DMPC), dipalmitoylphosphatidylcholine (DPPC), dipalmitoylphosphatidylglycerol (DPPG), phosphatidylethanolamine (PE), dipalmitoylphosphatidylethanolamine (DPPE), phosphatidylglycerol (PG), phosphatidylinositol (PI), monosialoganglioside and sphingomyelin (SPM); distearoylphosphatidylcholine (DSPC), and dimyristoylphosphatidylglycerol (DMPG), To prepare the liposomes the phospholipid is hydrated using distilled water or other suitable diluent. Note that because the different phospholipids have different phase transition temperatures the whole process must be performed at a temperature above the phase transition temperature of the phospholipid.

In the preferred embodiment of this invention the phospholipid selected is one that has a low phase transition temperature such as dioleoyl phosphatidylcholine (phase transition temperature −22 C), or egg phosphatidylcholine (phase transition temperature −10 C), or soy phosphatidylcholne (phase transition temperature −20 C). The procedure for preparing liposomes using a low phase transition temperature phospholipid is simple and straight-forward. Briefly, the phospholipid is hydrated with distilled deionized water, and the suspension is shaken and sonicated to form a suspension of multilamellar liposomes. The liposomes thus prepared will have a wide range in sizes from 100 nm to above 1,000 nm in diameter. The liposomes are extruded through a filter with a pore size that is about 1,000 nm to remove very large liposomes and any aggregates that may be present.

The pre-blocking liposomes thus prepared will be composed of multilamellar liposomes. The number of lamellae comprising the liposome can be reduced by extruding the preparation through a membrane with a defined pore size. This will yield a liposome preparation where there is a broad range in the sizes of the liposomes from 100-1,000 nm with most of the liposomes being between 400-600 nm. This invention teaches that this wide range in liposome size and their modal distribution around 400-600 nm will provide the most effective means of blocking the RES. Further, that the number of layers of lamellae that make up the pre-blocking liposome is irrelevant to its pre-blocking activity which is solely dependent on the size and composition of the outermost bilayer membrane of the liposome. This is because it is only the outermost layer that is exposed to be coated with opsonins and recognized and taken up by the RES.

This points out an important difference between the liposomes of this invention and conventional liposomes. While conventional liposomes and made to be of a uniform size that is generally below 200 nm diameter and often to be about 100 nm or less, the pre-blocking liposomes are made to be much larger and with a much larger variation in size. In the preferred embodiment of this invention there is a normal distribution with the bulk of the pre-blocking liposomes being around 400 nm-600 nm with a rapid decline in the number of liposomes in the upper and lower end of the distribution curve. Also note that there is no requirement that the pre-blocking liposomes fall within a tightly controlled uniform narrow range as is typically specified for conventional liposomes.

Another important difference between the pre-blocking liposomes of this invention and conventional liposomes is that cholesterol is not included in the pre-blocking liposome formulation. This is because in conventional liposomes cholesterol is used to stabilize the lipid bilayer of the liposome and thus prevent leakage of the drug from the interior of the liposome to the exterior medium. However, drug leakage is obviously not a concern for pre-blocking liposomes and therefore cholesterol is not included in its formulation. The absence of cholesterol may actually be of advantage as there are reports that leaky liposomes are more rapidly taken up by the RES than stabilized liposomes.

Another advantage of using large pre-blocking liposomes is that they are too large to extravasate into the tumor tissue. Many tumors are supplied by blood vessels that have an abnormal vasculature. These blood vessels have capillaries that have very large endothelial pores that can be as large as 400 nm. This feature can be used to advantage by conventional liposomal drugs that are smaller than 200 nm. These liposomal drugs are able to extravasate through the enlarged endothelial pores of the leaky blood capillaries supplying the tumor and concentrate within the tumor. This is known as “Enhanced Permeation and Retention (EPR)” effect. (Matsumura Y, Maeda H. 1986). The pre-blocking liposomes being too large to extravasate thru the enlarged endothelial pores are unable to block the liposomal drug from entering the tumor tissue. This is an important feature to consider in the situation where a saturation dosage of pre-blocking liposomes is administered and there is an excess of pre-blocking liposomes remaining in the blood when the liposomal drug is administered. The presence of excess pre-blocking liposomes in the blood will have a continued blocking effect on the RES while at the same time allowing the liposomal drug to extravasate and passively localize within the tumor.

In one embodiment of this invention an antioxidant such as alpha-tocopherol or butylated hydroxyl toluene (BHT) is added to the pre-blocking liposomal formulation in order to prevent oxidation and degradation of the phospholipid during storage.

Another method of preventing oxidation of the pre-blocking liposomes is to store them under an inert gas such as argon or nitrogen and to store them in the dark at 4 C or frozen at −20 C. For prolonged storage a preservative such as trehalose can be added and the pre-blocking liposomes lyophilized and stored at 4 C or at −20 C. The lyophilized pre-blocking liposomes can be reconstituted by adding distilled deionized water or a suitable diluent at a temperature above the phase transition temperature of the phospholipid and shaking the preparation. The rehydrated pre-blocking liposomes are filtered through a filter with a pore size of about 1,000 nm to remove any large aggregates that may have formed before it is administered to the patient.

In one embodiment of this invention the pre-blocking liposomes are pre-coated with opsonins before they are administered to the patient. Opsonins is the term used to describe the components of plasma that spontaneously coat the surface of foreign objects such as pathogens (e,g, bacteria) to facilitate their recognition and removal by the RES. Pre-coating the pre-blocking liposomes by incubating them with human plasma, or certain components of human plasma such as Immunoglobulin G (IgG) or complement C3 could enhance their recognition and uptake by the RES.

In contrast to conventional liposomal drugs where it is important to shield them from uptake and destruction by the RES the pre-blocking liposomes are expressly designed to do the opposite and be taken up by the RES. Typically, most of the pre-blocking liposomes are trapped by the RES soon after administration and almost all are trapped within the first hour or two. However, the blocking effect diminishes with time and is essentially gone 24 hours later. Therefore to take advantage of the RES blockade it is recommended that the therapeutic liposomal drug be given about 2 hours after administration of the pre-blocking liposomes.

With the development of stabilized “stealth” liposomes and other drug delivery systems that can extend the bioavailability of the (liposomal drug in the blood circulation for up to several days it may become necessary to extend the period of time that the RES is blocked. For example when the blocking effect of the first dose is wearing off and there is still a significant portion of the therapeutic liposomal drug still circulating in the blood a second dose of pre-blocking liposomes can be administered. The timing of the second dose will depend on the amount of therapeutic drug remaining in the blood. The precise timing of the second blocking dose will vary depending on the particular therapeutic drug being used.

One further benefit of administering pre-blocking liposomes is that it may mitigate certain adverse side-effects that develop during chemotherapy using drug incorporated nanocarriers. There are reports that repeated administration of drug nanocarriers over time may lead to the development of an adverse immune reaction to the administered drug nanocarrier. Administering pre-blocking liposomes will mitigate this reaction as any heightened reactivity of the RES will be directed to the pre-blocking liposomes and less to the subsequent administration of the therapeutic drug nanocarrier.

In one embodiment of this invention a pharmaceutical kit for preparing the pre-blocking liposomes is disclosed. This invention teaches a means of preparing the pre-blocking liposomes on site prior to administering them intravenously into the cancer patient. The kit is composed of the following components: a) one vial containing a standardized amount of lyophilized pre-blocking liposomes sealed under vacuum or under an inert gas; b) one vial of distilled deionized water or a physiological solution; c) one syringe; d) one filter unit and e) a package insert with information and instructions on the kit. The contents of the kit are packaged to exclude light and the kit is stored at 4 C.

It is important to note that the pre-blocking liposomes effect upon the RES is non-specific in nature. The blocking effect is not limited to optimizing only liposomal drugs but can be applied to optimizing all types of drug delivery systems that are predisposed to be recognized and trapped by the RES. This will include liposomes, micelles, dendrimers, nanoparticles, lipid nanospheres, nanocapsules, nanoemulsions, antibody drug conjugates and other nanosized drug delivery systems.

It will be obvious to those of skill in the art that there are various modifications and changes in the composition of the pre-blocking liposomes that can be made without departing from the teaching of this invention. Such changes are therefore considered to lie within the spirit and scope of this invention.

### REFERENCES

Abra, R. M., Hunt, C. A. Liposome disposition in vivo. Ill Dose and vesicle size effects. Biochim Biophys Acta. 1981; 666: 493-503
Abra, R. M., Hunt, C. A. Liposome disposition in vivo. iV: The interaction of sequential doses of liposomes having different diameters. Res Commun Chem Pathol Pharmacol 1982 April 36 (1):17-31
Bangham, A. D., et al. Diffusion of univalent ions across the lamellae of swollen phospholipids. J. Mol Biol. 1965 13: 238-252
Bangham, A. D. et al. Preparation and use of liposomes as models of biological membranes, in Korn, D. (Ed). Methods in Membrane Biology. Vol. 1. Plenum Press, New York. 1974: pp. 1-68.
Ellens, H. et al. Reversible depression of the reticuloendothelial system by liposomes. Biochem Biophys Acta. 1982; 714:479-485.
Gregoriadis, G. Ryman B. E. Liposomes as carriers of enzymes or drugs; a new approach to the treatment of storage diseases. Biochem J. 1971 124: 58P
Gregoriadis, G. The carrier potential of liposomes in biology and medicine. N Engl. J. Med. 1976. 295:703-710 and 765-770
Juliano, R. L., Stamp, D. Effect of particle size and charge on the clearance rate of liposomes and liposome encapsulated drugs. Biochem Biophys Res Commun. 1975; 63:651.
Kao, Y. J., Juliano, R. L. Interaction of liposomes with the reticuloendothelial system. Biochem Biophys Acta. 1981; 677: 453-461.
Lui T et al. RES blockade: A strategy for boosting efficiency of nanoparticle drug. Nanotoday 2015 Vol 10 Issue 1: 11-21
Matsumura Y, Maeda H. A new concept for macromolecular therapeutics in cancer chemotherapy; mechanism of tumoritropic accumulation of proteins and the antitumor agent smanes. Cancer research. 1986; 46 (12 Part 10: 6387-92
Sandeep K. et al. Liposomal drug delivery system—A comprehensive review. Int. J. Drug Dev. & Res. 2013. Vol 5 (4) 62-75

What is claimed is:

1. A method of increasing the safety and efficacy of a small molecule cancer drug incorporated in a nanocarrier by pre-blocking the reticuloendothelial system (RES) of the patient prior to administering the therapeutic drug; wherein the pre-blocking agent is an empty liposomal formulation composed solely of one or more phospholipids hydrated with distilled deionized water or a physiological solution and excluding cholesterol from the formulation; and wherein said empty liposomes are non-uniform in size and range from 100 nm to 1,000 nm in diameter, with most of the liposomes being between 400 nm to 600 nm in diameter; and wherein optionally, said empty liposomes are coated with opsonins in order to facilitate their uptake by the RES.

2. A method according to claim 1 wherein the one or more phospholipids used are selected from the following list: dioleoylphosphatidylcholine (DOPC), egg phosphatidylcholine (EPC), hydrogenated egg phosphatidylcholine (HEPC), soy phosphatidylcholine (SPC), hydrogenated soy phosphatidylcholine (HSPC), dimyristoylphosphatidylcholine (DMPC), dipalmitoyl phosphatidylcholine (DPPC), dipalmitoylphosphatidylglycerol (DPPG), phosphatidylethanolamine (PE), phosphatidylglycerol (PG), phosphatidylinositol (PI), monosialoganglioside and sphingomyelin (SPM); distearoylphosphatidylcholine (DSPC), and dimyristoylphosphatidylglycerol (DMPG),

3-8. (canceled)
